(19) United States
(12) Patent Application Publication
(10) Pub. No.: US 2019/0192686 A1
(43) Pub. Date: Jun. 27, 2019

(54) TARGETED NANODROPLET EMULSIONS FOR TREATING CANCER

(71) Applicant: The University of Louisville Research Foundation,, Louisville, KY (US)

(72) Inventors: Mohammad Tariq Malik, Prospect, KY (US); Jonathan A. Kopechek, LaGrange, KY (US); Paula J. Bates, Louisville, KY (US)

(21) Appl. No.: 16/322,369

(22) PCT Filed: Aug. 2, 2017
(86) PCT No.: PCT/US2017/045169

§ 371 Date: Jan. 31, 2019

Related U.S. Application Data
(60) Provisional application No. 62/370,137, filed on Aug. 2, 2016.

(51) Int. Cl. A61K 47/69; A61K 49/22; A61K 47/54; A61K 47/68; A61K 31/122; A61K 31/337

(52) U.S. Cl. CPC A61K 47/6909; A61K 49/227; A61K 47/549; A61K 47/6843; A61K 31/122; A61K 31/337; A61K 47/6803

(57) ABSTRACT

A micelle, comprises a first phospholipid, a second phospholipid, a targeting agent, conjugated to the first phospholipid, a perfluorocarbon, and a therapeutically active compound. The first phospholipid and the second phospholipid form a shell enclosing the perfluorocarbon and the therapeutically active compound. The targeting agent comprises an anti-nucleolin agent, and the therapeutically active compound comprises a chemotherapeutic agent and/or a cytotoxic agent. An emulsion may be formed, comprising a plurality of the micelles, and continuous aqueous phase. A pharmaceutical composition for treating cancer may be prepared, comprising the emulsion, and a pharmaceutically acceptable carrier. A method of treating cancer includes administering an effective amount of the pharmaceutical composition to a patient in need thereof.

### BACKGROUND

Standard clinical treatments for cancer patients include surgery, radiation, and chemotherapy, Administration of chemotherapeutic drugs has been used for cancer treatment since the 1940s but targeted anti-cancer therapies have only recently been developed [10, 11]. Currently, conventional chemotherapy drugs are typically delivered systemically and cause serious side effects in other organs, including reduced immune activity and damage to organs such as the heart and kidneys [12]. Therefore, the maximum dose that can be administered is limited. To address this problem, targeted delivery strategies are in development to increase the efficacy of chemotherapy while reducing systemic toxicity. One such method of targeted delivery utilizes targeting agents that bind to nucleolin.

Nucleolin [8] is an abundant, non-ribosomal protein of the nucleolus, the site of ribosomal gene transcription and packaging of pre-ribosomal RNA. This 710 amino acid phosphoprotein has a multi-domain structure consisting of a histone-like N-terminus, a central domain containing four RNA recognition motifs and a glycine/arginine-rich C-terminus, and has an apparent molecular weight of 110 kD. While nucleolin is found in every nucleated cell, the expression of nucleolin on the cell surface has been correlated with the presence and aggressiveness of neoplastic cells [3].

The correlation of the presence of cell surface nucleolin with neoplastic cells has been used for methods of determining the neoplastic state of cells by detecting the presence of nucleolin on the plasma membranes [3]This observation has also provided new cancer treatment strategies based on administering compounds that specifically target nucleolin [4].

Nucleic acid aptamers are short synthetic oligonucleotides that fold into unique three-dimensional structures that can be recognized by specific target proteins. Thus, their targeting mechanism is similar to monoclonal antibodies, but they may have substantial advantages over these including more rapid clearance in vivo, better tumor penetration, non-immunogenicity, and easier synthesis and storage.

Guanosine-rich oligonucleotides (GROs) designed for triple helix formation are known for binding to nucleolin. This ability to bind nucleolin has been suggested to cause their unexpected ability to effect antiproliferation of cultured prostate carcinoma cells [6]. The antiproliferative effects are not consistent with a triplex-mediated or an antisense mechanism, and it is apparent that GROs inhibit proliferation by an alternative mode of action. It has been surmised that GROs, which display the propensity to form higher order structures containing G-quartets, work by an aptamer mechanism that entails binding to nucleolin due to a shape-specific recognition of the GRO structure: the binding to cell surface nucleolin then induces apoptosis. The antiproliferative effects of GROs have been demonstrated in cell lines derived from prostate (DU145), breast (MDA-MB-231, MCF-7), or cervical (HeLa) carcinomas and correlates with the ability of GROs to bind cell surface nucleolin [6].

AS1411, a GRO nucleolin-binding DNA aptamer that has antiproliferative activity against cancer cells with little effect on non-malignant cells, was previously developed. AS1411 uptake appears to occur by macropinocytosis in cancer cells, but by a nonmacropinocytic pathway in nonmalignant cells, resulting in the selective killing of cancer cells, without affecting the viability of nonmalignant cells [9]. AS1411 was the first anticancer aptamer tested in humans and results from clinical trials of AS1411 (including Phase II studies in patients with renal cell carcinoma or acute myeloid leukemia) indicate promising clinical activity with no evidence of serious side effects. Despite a few dramatic and durable clinical responses, the overall rate of response to AS1411 was low, possibly due to the low potency of AS1411.

Ultrasound-responsive nanodroplet emulsions have been used for targeted delivery of molecular therapeutics [47]. In one study, an aptamer (sgc8c) was used to target nanodroplets loaded with doxorubicin to CCRF-CEM cells. High-intensity focused ultrasound (HIFU) was introduced to trigger targeted acoustic droplet vaporization, to cause the doxorubicin to chemically treat the cells and cause mechanical damage to the cells.

### SUMMARY

In a first aspect, the invention is a micelle, comprising a first phospholipid, a second phospholipid, a targeting agent, conjugated to the first phospholipid, a perfluorocarbon, and a therapeutically active compound. The first phospholipid and the second phospholipid form a shell enclosing, the perfluorocarbon and the therapeutically active compound, the targeting agent comprises an anti-nucleolin agent, and the therapeutically active compound comprises a chemotherapeutic agent and/or a cytotoxic agent.

In a second aspect, the present invention is an emulsion, comprising a plurality of the micelles, and a continuous aqueous phase.

In a third aspect, the present invention is a pharmaceutical composition for treating cancer, comprising the emulsion, and a pharmaceutically acceptable carrier.

In a fourth aspect, the present invention is a method of treating cancer, comprising administering an effective amount of the pharmaceutical composition to a patient in need thereof.

In a fifth aspect, the present invention is a method of imaging cancer or a tumor, comprising (1) administering the micelle or emulsion to a patient, and imaging the cancer or tumor, with ultrasound.

### DEFINITIONS

The term ‘CRO’ means a control aptamer

An “anti-nucleolin agent” includes any molecule or compound that interacts with nucleolin. Such agents include, for example, anti-nucleolin antibodies, peptides. pseudopeptides aptamers such GROs and nucleolin targeting proteins.

Tumors and cancers include solid, dysproliferative tissue changes and diffuse tumors. Examples of tumors and cancers include melanoma, lymphoma, plasmocytoma, sarcoma, glioma, thymoma, leukemia, breast cancer, prostate cancer, colon cancer, liver cancer, esophageal cancer, brain cancer, lung cancer, ovary cancer, endometrial cancer, bladder cancer, kidney cancer, cervical cancer, hepatoma, and other neoplasms. For more examples of tumors and cancers, see, for example Stedman [1].

“Treating a tumor” or “treating a cancer” means to significantly inhibit growth and/or metastasis of the tumor or cancer, and/or killing cancer cells. Growth inhibition can be indicated by reduced tumor volume or reduced occurrences of metastasis. Tumor growth can be determined, for example, by examining the tumor volume via routine procedures (such as obtaining two-dimensional measurements with a dial caliper). Metastasis can be determined by inspecting for tumor cells in secondary sites or examining the metastatic potential of biopsied tumor cells in vitro.

A “chemotherapeutic agent” is a chemical compound that can be used effectively to treat cancer in humans.

A “pharmaceutically acceptable carrier” includes any and all solvents, dispersion media, coatings, antibacterial and antifungal agents, isotonic and absorption delaying agents which are compatible with pharmaceutical administration. Preferred examples of such carriers or diluents include water, saline, Ringer's solutions and dextrose solution. Supplementary active compounds can also be incorporated into the compositions.

“Medicament,” “therapeutic composition,” and “pharmaceutical composition” are used interchangeably to indicate a compound, matter, mixture or preparation that exerts a therapeutic effect in a subject, which is preferably sterile and ready for use, for example in a unit dosage form,

“Therapeutically active compound” is an active agent used to treat a disease or condition, or exert an effect on cells of a patient, such as a chemotherapeutic agent or a cytotoxic agent.

“Nanodroplets” or “nanoemulsions” are a type of micelle, composed of a biocompatible phospholipid shell encapsulating an inert, non-toxic perfluorocarbon such as perfluoropentane. The micelle has a single lipid layer, and does not have a lipid bilayer.

Particle size means average particle diameter as determined by a particle size analyzer using light scattering, for example, a NanoBrook 90Plus Particle Size Analyzer.

The amounts and ratios of compositions described herein are all by weight, unless otherwise stated.

### BRIEF DESCRIPTION OF THE DRAWINGS

The invention can be better understood with reference to the following drawings and description

FIG. 1 illustrates AS1411-conjugated micelles targeting tumors.

FIG. 2A illustrates the AS1411 sequence, with linker regions (5′TTTTTT, 3′TTT) and with a thiol group on the 3′ end.

FIG. 2B illustrates micelles with aptamers attached to a maleimide-containing phospholipid shell.

FIG. 2C illustrates micelles with a phospholipid shell, a targeting agent, a perfluorocarbon and a therapeutically active compound.

FIG. 3 illustrates a graph showing dynamic light scattering measurements indicating minimal change in size distribution of AS1411-conjugated micelles up to 48 hr. maleimide-lipid micelles without aptamer.

FIG. 4 illustrates the mean fluorescence intensity of cancer cells incubated with fluorescent AS1411-conjugated micelles as a function of (A) time and (B) dose after 72 hr (n=10). Representative fluorescence microscopy images at each (C) time point and (D) dose indicate uptake of micelles compared to the initial control sample maleimide-lipid micelles with CRO (control aptamer).

FIG. 5A illustrates confocal microscopy images of human breast cancer cells (MDA-MB-231) with no treatment.

FIG. 5B illustrates confocal microscopy images of human breast cancer cells (MDA-MB-231) indicating uptake of fluorescent AS1411-conjugated micelles after 4 hr incubation with micelles. (Red: Cy5-labeled AS1411, Green: FITC-labeled lipid, Blue: DAPI nuclear stain).

FIG. 5C illustrates confocal microscopy images of human breast cancer cells (MDA-MB-231) indicating uptake of fluorescent AS1411-conjugated micelles after 24 hr incubation with micelles. (Red: Cy5-labeled AS1411, Green: FITC-labeled lipid, Blue: DAPI nuclear stain).

FIG. 6 illustrates the fluorescence intensity in human breast cancer cells (MDA-MB-231) measured using flow cytometry measured after incubation for 4 hr with FITC-labeled micelles and Cy5-AS1411 labeled micelles.

FIG. 7A illustrates the cytotoxicity of thymoquinone (TQ)-loaded micelles as concentration increases in MDA-MB-231 cells,

FIG. 7B illustrates the cytotoxicity of thymoquinone (TQ)-loaded micelles as concentration increases in HCC1395 cells.

FIG. 7C illustrates the cytotoxicity of thymoquinone (TQ)-loaded micelles as concentration increases in A549 cells.

FIG. 8 illustrates aptamer loading efficiency of micelles with and without maleimide.

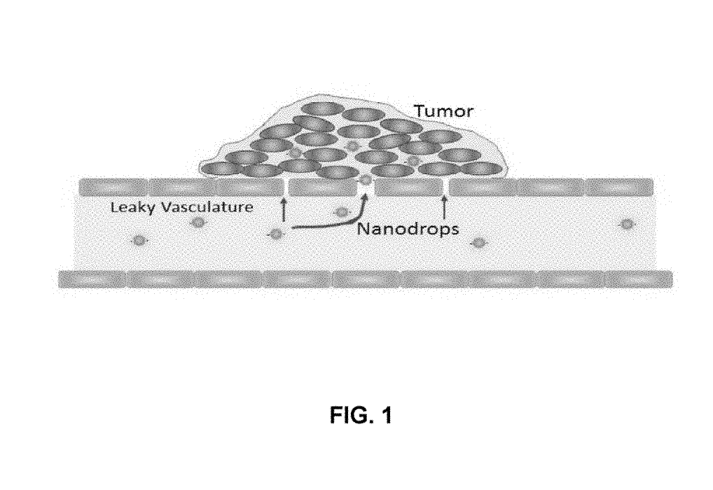

### DETAILED DESCRIPTION

The present invention makes use of perfluorocarbon-based micelles that are conjugated to targeting agents, causing an antiproliferative effect on cancers and tumors. The micelles contain a therapeutically active compound. The targeting agent targets the micelles to cancer cells or tumors, by binding to nucleolin. Once the micelles enter the tumor area, ultrasound may be used to induce a phase change of the perfluorocarbon, from liquid to gas, causing the micelles to rupture, and release the therapeutically active compound. Optionally, the use of a light-absorbing dye, in conjunction with a light delivery method (such as a laser), may be used to induce phase change of the perfluorocarbon and cause the rupture of the micelles. The micelles may enter the cells via endocytic pathways where their components are metabolized and the therapeutically active compound is released. Furthermore, the micelles may be used in ultrasound imaging, where the formation of a gas phase within the micelles enhances the contrast of the ultrasound image.

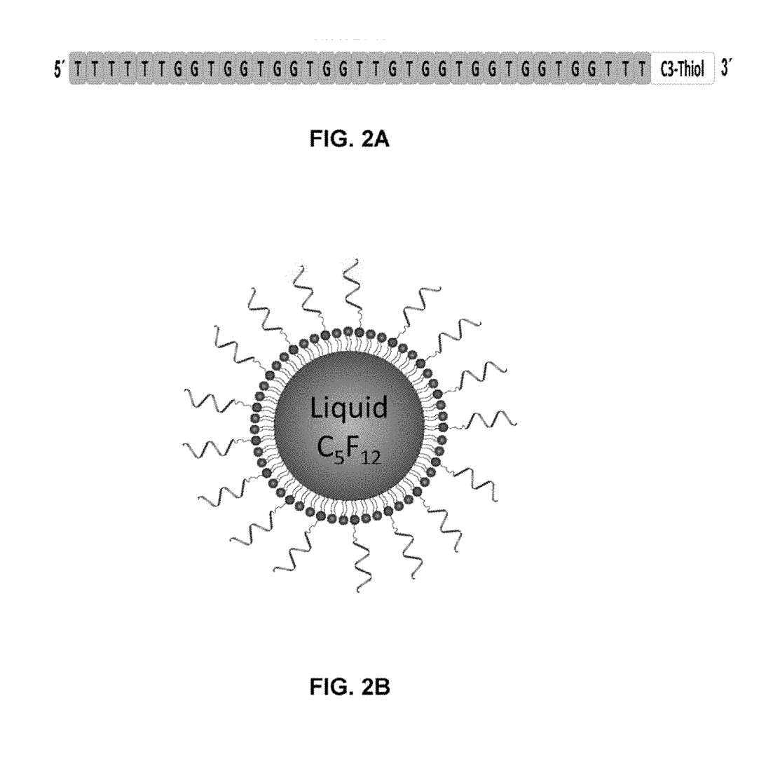

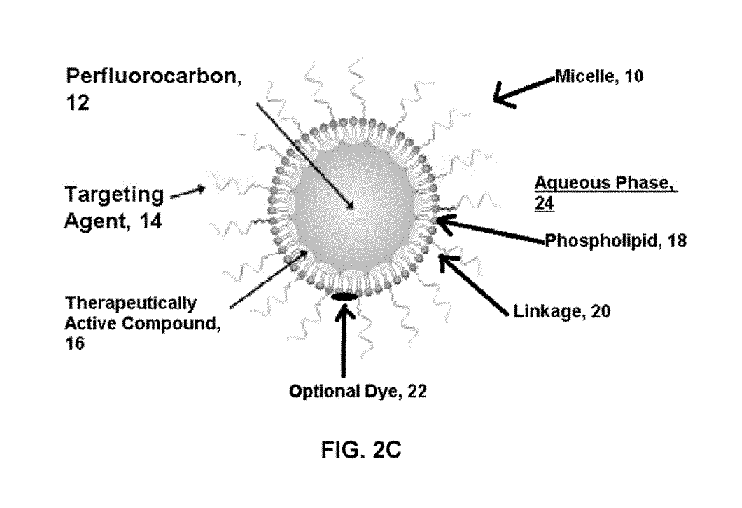

FIG. 2C illustrates an emulsion, including a micelle, 10, conjugated to a targeting agent, 14, and includes a perfluorocarbon, 12, and a therapeutically active compound, 16. The micelle has one or more phospholipids, 18, which form the shell that surrounds the perfluorocarbon. The targeting agent, 14, is conjugated to some of the phospholipids through a linkage, 20. The micelles are present within an aqueous phase, 24, which form the emulsion. Optionally, the micelle may include a dye, 22, which may be conjugated to some of the phospholipids through a linkage (as illustrated), or may be present with the perfluorocarbon and therapeutically active compound inside the shell.

Each phospholipid has a hydrophilic phosphate head and lipophilic tail, In the micelles the lipophilic tails face the inside, while the phosphate heads face the outside. A first phospholipid is conjugated to the targeting agent. A second phospholipid is not conjugated. Optionally, a third phospholipid may be conjugated to a dye, Additional phospholipids, as well as other compounds such as cholesterol, may be present and form the shell. Examples include phospholipids (such as 1,2-dipalmitoyl-sn-glycero-3-phosphocholine (DPPC)), PEGylated phospholipids (such as 1,2-distearoyl-sn-glycero-3-phosphoethanolamine-N-[amino(polyethylene glycol)-2000] (DSPE-PEG2000)), phospholipids having a linking agent (such as 1,2-distearoyl-sn-glycero-3-phosphoethanolamine-N[maleimide(polyethylene glycol)2000] (DSPE-PEG2000-maleimide)), and phospholipids having a dye (such as 1,2-distearoyl-sn-glycero-3-phosphoethanolamine (DSPE-PEG2000-FITC)). In addition to the phospholipid shell, a biocompatible fluorosurfactant (such as perfluoropolyethers with PEG or Tris) could potentially be added for improved long-term stability.

To form the micelles, a phospholipid solution in an aqueous phase is prepared. The phospholipid solution contains at least a first phospholipid and a second phospholipid; additional phospholipids may be present. The first phospholipid has a phosphate head connected to a first linking agent, such as a maleimide. The therapeutically active compound may then be added. Next, the targeting agent with a protected second linking agent is deprotected and immediately combined with the phospholipid solution to allow for conjugation between the first phospholipid and the targeting agent. Then, a perfluorocarbon is combined with this solution, and the resulting emulsion is sonicated. The emulsion may then be centrifuged to remove unbound elements from the micelles.

Alternatively, the different phospholipids which will form the micelles are simply mixed together in an aqueous phase (including the phospholipids conjugated to the targeting agent, and optionally phospholipid conjugated to the dye), together with therapeutically active compound and the perfluorocarbon, and sonicated to form the micelles and the emulsion. The emulsion is may then be centrifuged to remove unbound elements from the micelles. Such techniques are also described in [41], [42] and [43].

To make the micelles generally uniform in size, the micelles may be extruded through a membrane, having a specific pore size, such as a membrane having 0.2 μm or 0.1 μm diameter openings. The micelles are extruded through the membrane multiple times, such as 1-20 times, more preferably 5-15 times, to produce micelles having a more uniform size and a narrower size distribution. The micelles may have an average diameter of 50-500 nm, as measured by a particle size analyzer, more preferably an average diameter of 125-300 nm, and most preferably an average diameter of 150-250 nm. Micelles with an average diameter of 100-200 nm may also be preferred. Preferably, the standard deviation of the diameter of the micelles is at most 50 nm, more preferably at most 25 nm, even more preferably at most 10 nm, and most preferably at most 5 nm.

When a majority of micelles have a diameter (taking into account average diameter and the standard deviation of the diameter) of less than 300 nm, more preferably less than 250 nm, then a majority of the micelles can enter inside the cells when the targeting agent is an anti-nucleolin agent. Using micelles having an average diameter of at most 200 nm, such as, 100-200 nm, may be more desirable.

The perfluorocarbon used in the micelle may be perfluoropentane, perfluorohexane, perfluoroheptane, perfluorooctane, and perfluorononane. Preferably the perfluorocarbon is perfluoropentane. Ultrasound or light can be used to initiate boiling of the perfluorocarbon in the micelle and release the therapeutically active compound. While some perfluorocarbons, such as perfluoropentane, have a boiling point lower than human body temperature, the perfluoropentane remains in the liquid phase after being introduced into a human subject, because the pressure inside the micelle raises the boiling point of the perfluorocarbon; The perfluorocarbon acts like a superheated fluid, transforming into the gas phase upon application of the ultrasound in a sudden and complete phase change. For perfluorocarbons with higher boiling points, such as perfluorooctane, ultrasound, such high-intensity focused ultrasound (HIFU) may not be sufficient to increase the temperature of the micelles to induce a phase change. In these instances, a light-absorbing dye, such as a cyanine dye or FITC, may be included as part of the micelle, and light may be used to induce a phase change from liquid to gas in the micelle, for example infrared light, visible light or UV light.

The therapeutically active compound may be a chemotherapeutic agent used for the treatment of cancer. Examples of commonly used chemotherapeutic agents include vinorelbine (Navelbine®), mytomycin, camptothecin, cyclyphosphamide (Cytoxin®), methotrexate, tamoxifen citrate, 5-fluorouracil, innotecan, doxorubicin, flutamide, paclitaxel (Taxol®), docetaxel, vinblastine, imatinib mesylate (Gleevec®), anthracycline, letrozole, arsenic trioxide (Trisenox®), anastrozole, triptorelin pamoate, ozogamicin, irinotecan hydrochloride (Camptosar®), leuprolide acetate implant (Viadur), bexarotene (Targretin®), exemestane (Aromasin®), topotecan hydrochloride (Hycamtin®), gemcitabine (Gemzar®), daunorubicin hydrochloride (Daunorubicin HCL®), toremifene citrate (Fareston), carboplatin (Paraplatin®), cisplatin (Platinol® and Platinol-AQ®) oxaliplatin and any other platinum-containing oncology drug, trastuzumab (Herceptin®), lapatinib (Tykerb®), gefitinb (Iressa®), cetuximab (Erbitux®), panitumumab (Vectibix®), temsirolimus (Torisel®), everolimus (Afinitor®), vandetanib (Zactima™), vemurafenib (Zelbora™), crizotinib (Xalkori®), vorinostat(Zolinza®), and, bevacizumab (Avastin®). Preferably the chemotherapeutic agent is hydrophobic, such as paclitaxel, temsirolimus, everolimus, dactinomycin, etoposide, teniposide, cyclophosphamide, rapamycin, camptothecin, or thymoquinone. Preferably the chemotherapeutic agent is thymoquinone or paclitaxel.

It is well known that lipophilic and lipophobic properties of chemotherapeutic agents may be adjusted using well known chemical techniques, such as esterification. Multiple chemotherapeutic agents may be administered together, such as 2 or 3 chemotherapeutic agents, either by producing micelles with multiple chemotherapeutic agents, or by mixing batches of micelles, each containing a different chemotherapeutic agent.

The therapeutically active compound may be a cytotoxic agent, such as pore-forming toxins (PFT), SN-38, radionuclides or magnetic spin-vortex discs, which are magnetized only when a magnetic field is applied to avoid self-aggregation that can block blood vessels, begin to spin when a magnetic field is applied, causing membrane disruption of target cells.

The targeting agent is preferably an anti-nucleolin agent. Anti-nucleolin agents may include antibodies, proteins, GROs, aptamers, or other compounds that bind to nucleolin. Targeting agents include aptamers, such as GROs. Examples of aptamers include guanosine-rich oligonucleotides (GROs). Examples of suitable oligonucleotides and assays are also given in Miller et al. [7]. Characteristics of GROs include:
(1) having at least 1 GGT motif,
(2) preferably having 4-100 nucleotides, although GROs having many more nucleotides are possible,
(3) optionally having chemical modifications to improve stability.

Especially useful GROs form G-quartet structures, as indicated by a reversible thermal denaturation/renaturatian profile at 295 nm. Preferred GROs also compete with a telomere oligonucleotide for binding to a target cellular protein in an electrophoretic mobility shift assay [6]. In some cases, incorporating the GRO nucleotides into larger nucleic acid sequences may be advantageous; for example, to facilitate binding of a GRO nucleic acid to a substrate without denaturing the nucleolin-binding site. Examples of oligonucleotides are shown in Table 1; preferred oligonucleotides include SEQ IDs NOs: 1-7; 9-16; 19-30 and 31 from Table 1. Most preferably, the targeting agent is AS1411. AS1411 advantages over other aptamers include increased internalization into the cancer or tumor cells and near-universal targeting of various tumor types.

TABLE 1
Non-antisense GROs that bind nucleolin and non-binding
controls^{1,2,3}.

| GRO | Sequence | SEQ ID NO. |
|---|---|---|
| GRO29A¹ | tttggtggtg gtggttgtgg tggtggtgg | 1 |
| GRO29-2 | tttggtggtg gtggttttgg tggtggtgg | 2 |
| GRO29-3 | tttggtggtg gtggtggtgg tggtggtgg | 3 |
| GRO29-5 | tttggtgatg gtggtttggg tggtggtgg | 4 |
| GRO29-13 | tggtggtggt ggt | 5 |
| GRO14C | ggtggttgtg gtgg | 6 |
| GRO15A | gttgtttggg gtggt | 7 |
| GRO15B² | ttgggggggg tgggt | 8 |
| GRO25A | ggttggggtg ggtggggtgg gtggg | 9 |
| GRO26B¹ | ggtggtggtg gttgtggtgg tggtgg | 10 |
| GRO28A | tttggtggtg gtggttgtgg tggtggtg | 11 |
| GRO28B | tttggtggtg gtggtgtggt ggtggtgg | 12 |
| GRO29-6 | ggtggtggtg gttgtggtgg tggtggttt | 13 |
| GRO32A | ggtggttgtg gtggttgtgg tggttgtggt gg | 14 |
| GRO32B | tttggtggtg gtggttgtgg tggtggtggt tt | 15 |
| GRO56A | ggtggtggtg gttgtggtgg tggtggttgt | 16 |
| | ggtggtggtg gttgtggtgg tggtgg | |
| CRO | tttcctcctc ctccttctcc tcctcctcc | 18 |
| GRO A | ttagggttag ggttagggtt aggg | 19 |
| GRO B | ggtggtggtg g | 20 |
| GRO C | ggtggttgtg gtgg | 21 |
| GRO D | ggttggtgtg gttgg | 22 |
| GRO E | gggttttggg | 23 |
| GRO F | ggttttggtt ttggttttgg | 24 |
| GRO G¹ | ggttggtgtg gttgg | 25 |
| GRO H¹ | ggggttttgg gg | 26 |
| GRO I¹ | gggttttggg | 27 |
| GRO J¹ | ggggttttgg ggttttgggg ttttgggg | 28 |
| GRO K¹ | ttggggttgg ggttggggtt gggg | 29 |
| GRO L¹ | gggtgggtgg gtgggt | 30 |
| GRO M¹ | ggttttggtt ttggttttgg ttttgg | 31 |
| GRO N² | tttcctcctc ctccttctcc tcctcctcc | 32 |
| GRO O² | cctcctcctc cttctcctcc tcctcc | 33 |
| GRO P² | tggggt | 34 |
| GRO Q² | gcatgct | 35 |
| GRO R² | gcggtttgcg g | 36 |
| GRO S² | tagg | 37 |
| GRO T² | ggggttgggg tgtggggttg ggg | 38 |

¹Indicates a good plasma membrane nucleolin-binding GRO.
²Indicates a nucleolin control (non-plasma membrane nucleolin binding).
³GRO sequence without ¹ or ² designatioris have some anti-proliferative activity.

Any antibody that binds nucleolin may also be used. In certain instances, monoclonal antibodies are preferred as they bind single, specific and defined epitopes. In other instances, however, polyclonal antibodies capable of interacting with more than one epitope on nucleolin may be used. Many anti-nucleolin antibodies are commercially available, and are otherwise easily made. Table 2 list a few commercially available anti-nucleolin antibodies.

TABLE 2
commercially available anti-nucleolin antibodies

| | | Antigen |
|---|---|---|
| Antibody | Source | source |
| p7-1A4 Mouse monoclonal antibody | Developmental Studies | Xenopus |
| (mAb) | Hybridoma Bank | laevis oocytes |
| Sc-8031 mouse mAb | Santa Cruz Biotech | human |
| Sc-9893 goat polyclonal Ab (pAb) | Santa Cruz Biotech | human |
| Sc-9892 goat pAb | Santa Cruz Biotech | human |
| Clone 4E2 mouse mAb | MBL International | human |
| Clone 3G4B2 mouse mAb | Upstate Biotechnology | dog (MDCK |
| | | cells) |
| Nucleolin, Human (mouse mAb) | MyBioSource | human |
| Purified anti-Nucleolin-Phospho, | BioLegend | human |
| Thr76/Thr84 (mouse mAb) | | |
| Rabbit Polyclonal Nucleolin Antibody | Novus Biologicals | human |
| Nucleolin (NCL, C23, FLJ45706, | US Biological | human |
| FLJ59041, Protein C23) Mab Mo xHu | | |
| Nucleolin (NCL, Nucl, C23, FLJ45706, | US Biological | human |
| Protein C23) Pab Rb xHu | | |
| Mouse Anti-Human Nucleolin Phospho- | Cell Sciences | human |
| Thr76/Thr84 Clone 10C7 mAb | | |
| Anti-NCL/Nucleolin (pAb) | LifeSpan Biosciences | human |
| NCL purified MaxPab mouse polyclonal | Abnova | human |
| antibody (B02P) | | |
| NCL purified MaxPab rabbit polyclonal | Abnova | human |
| antibody (D01P) | | |
| NCL monoclonal antibody, clone 10C7 | Abnova | human |
| (mouse mAb) | | |
| Nucleolin Monoclonal Antibody (4E2) | Enzo Life Sciences | human |
| (mouse mAb) | | |
| Nucleolin, Mouse Monoclonal Antibody | Life Technologies | human |
| | Corporation | |
| NCL Antibody (Center E443) (rabbit | Abgent | human |
| pAb) | | |
| Anti-Nucleolin, clone 3G4B2 (mouse | EMD Millipore | human |
| mAb) | | |
| NCL (rabbit pAb) | Proteintech Group | human |
| Mouse Anti-Nucleolin Monoclonal | Active Motif | human |
| Antibody, Unconjugated, Clone | | |
| 3G4B20 | | |
| Nsr1p - mouse monoclonal | EnCor Biotechnology | human |
| Nucleolin (mouse mAb) | Thermo Scientific Pierce | human |
| | Products | |
| Nucleolin [4E2] antibody (mouse mAb) | GeneTex | human |

Human antibodies, such as those described in U.S. Pat. No. 9,260,517 [44] may also be used.

Nucleolin targeting proteins are proteins, other than antibodies, that specifically and selectively bind nucleolin. Examples include ribosomal protein S3, tumor-homing F3 peptides and myosin H9 (a non-muscle myosin that binds cell surface nucleolin of endothelial cells in angiogenic vessels during tumorigenesis).

The targeting agent and/or dye may be conjugated to the phospholipid by using various methods and chemical techniques that form a linkage. The targeting agent may be attached by thioether linkage (thiol-maleimide) as described in the examples, where a thiol group is present on the targeting agent, and a maleimide is present on the phospholipid. The thiol group may be deprotected using a reducing agent, and the thiol and maleimide can conjugate together. Other mechanisms for conjugation include biotin-streptavidin bridge, amide linkage (for example reacting NHS ester with primary amine), a hydrazone linkage, and clik-chemistry. Preferably the attachment method occurs via a thiol-maleimide reaction.

The micelles, in the form of an emulsion, may be used as a medicament. A pharmaceutical composition is formulated to be compatible with its intended route of administration, including intravenous, intradermal, subcutaneous, oral, inhalation, transdermal, transmucosal, and rectal administration. Solutions and suspensions used for parenteral, intradermal or subcutaneous application can include a sterile diluent, such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide.

Pharmaceutical compositions suitable for injection include sterile aqueous solutions or dispersions for the extemporaneous preparation of sterile injectable solutions or dispersion. For intravenous administration, suitable carriers include physiological saline, bacteriostatic water, CREMOPHOR EL® (BASF; Parsippany, N.J.) or phosphate buffered saline (PBS). in all cases, the composition must be sterile and should be fluid so as to be administered using a syringe, Such compositions should be stable during manufacture and storage and are preferably preserved against contamination from microorganisms such as bacteria and fungi. The carrier can be a dispersion medium containing, for example, water, polyol (such as glycerol, propylene glycol, and liquid polyethylene glycol), and other compatible, suitable mixtures. Various antibacterial and anti-fungal agents, for example, parabens, chlorobutanol, phenol, ascorbic acid, and thimerosal, can contain microorganism contamination. Isotonic agents such as sugars, polyalcohols, such as mannitol, sorbitol, and sodium chloride can be included in the composition. Compositions that can delay absorption include agents such as aluminum monostearate and gelatin.

Sterile injectable solutions can be prepared by incorporating the active agents, and other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

In the treatment of cancer, an appropriate dosage level of the therapeutic agent will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. The compounds may be administered on a regimen of 1 to 4 times per day, preferably once per day prior to RT. Administration by continuous infusion is also, possible. All amounts and concentrations of anti-nucleolin oligonucieotide conjugated gold nanoarticles are based on the amount or concentration of anti-nucleolin oligonucleotide only.

Pharmaceutical preparation may be pre-packaged in ready-to-administer form, in amounts that correspond with a single dosage, appropriate for a single administration referred to as unit dosage form. Unit dosage forms can be enclosed in ampoules, disposable syringes or vials made of glass or plastic.

However, the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the patient undergoing therapy.

The medicaments of the present invention may be administered in combination with other cancer treatments such as chemotherapy, hyperthermia, gene therapy and photodynamic therapy.

A patient having cancer or a tumor, or suspected of having cancer or a tumor, such as a human, monkey, dog, cat, rabbit, cow, pig, goat, guinea pig, mouse, rat or sheep, may be treated by administration of the medicament. The patient may then be examined to determine if the administration has been effect to treat the cancer or tumor. Further administration to the patient may be desirable to further treat the cancer or tumor.

### EXAMPLES

### Example 1

### Materials and Methods:

### Synthesis of AS1411-Conjugted Drug-Loaded Nanodroplets

AS1411-conjugated nanodroplets loaded with thymoquinone were synthesized. The nanodroplets were composed of a perfluorocarbon core surrounded by a lipid shell. Thymoquinone, which is highly hydrophobic, incorporates within the lipid shell and thiolated-AS1411 was conjugated to maleimide-lipids (FIG. 2C). Phospholipids were obtained from Avanti Polar Lipids (Alabaster, Ala., USA). Nanodroplets were composed of 1,2-dipalmitoyl-sn-glycero-3-phosphocholine (DPPC), 1,2-distearoyl-sn-glycero-3-phosphoethanolamine-N-[amino(polyethylene glycol)-2000] (DSPE-PEG2000), and 1,2-distearoyl-sn-glycero-3-phosphoethanolamine-N-[maleimide(polyethylene glycol)-2000] (DSPE-PEG2000-maleimide) in a 96:2:2 molar ratio. For fluorescent studies 1,2-Distearoyl-sn-Glycero-3-Phosphoethanolamine (DSPE-PEG2000-FITC) was added instead of DSPE-PEG2000. Lipids were dissolved in chloroform and the solvent was evaporated under argon. The dry lipid film was rehydrated in phosphate buffered saline (PBS) to a concentration of 2.3 mg/mL. Thymoquinone (Sigma-Aldrich, St. Louis, Mo., USA) was added to the lipid solution at a concentration of 8 mg/mL and sonicated at 60% amplitude for 30 seconds with a sonicator to disperse the drug and lipid. Thiolated aptamers (50 μM of AS1411 or CRO, the negative control) were deprotected with 10 mM of (tris(2-carboxyethyl)phosphine) (TCEP) for 1 hour and immediately added to lipid solutions for overnight incubation at 4° C. to allow conjugation of aptamers to lipid via thiol-maleimide reaction.

To produce nanodroplets, perfluoropentane (Fluoromed, Round Rock, Tex., USA) was added to lipid solutions at 40% v/v and sonicated at 60% amplitude in an ice bath for 5 minutes in pulsed mode (20 s on, 40 s off, 1 min 40 s total sonication duration). Following sonication, the emulsion was centrifuged at 2000 g for 3 min and the supernatant was aspirated to remove lipid/drug/aptamer not bound to droplets. The pellet of droplets was resuspended and diluted 5-fold in PBS, followed by extrusion 10 times through a 0.2-μm membrane (Mini-extruder, Avanti Polar Lipids). The size distribution of the nanoemulsion was measured using a Particle Size Analyzer (Brookhaven Instruments, Holtsville, N.Y., USA). Thymoquinone loading was quantified with a NanoDrop One (Thermo Scientific, Waltham, Mass., USA) using the absorbance at 260 nm after subtracting the contribution from lipids and nanodroplets alone on the absorbance.

### Microscopy Imaging of Cellular Uptake

Fluorescent microscopy uptake studies were conducted using FITC-labeled, AS1411-conjugated nanodroplets. Images were acquired using an EVOS FL digital fluorescence microscope (Advanced Microscopy Group, Mill Creek, Wash., USA). Human A549 lung cancer cells were plated for 48 hr at a density of 4,000 cells/cm² in glass cell culture dishes (FluoroDish, World Precision Instruments, Sarasota, Fla. USA). AS1411-conjugated fluorescent nanodroplet emulsions were added to cells at various doses (0, 40×, 20×, 8×, 4×, and 2× dilutions) and incubated for various amounts of time (0, 1, 4, 24, 48, and 72 hr) at a 20× dilution. Slides were washed with HBSS, fixed with 3.5% paraformaldehyde, stained with 0.05% Hoechst 33342 for 5 minutes at room temperature to detect nuclei, washed twice, and mounted (ClearMount, Invitrogen, Frederick, Md., USA) for at least 3 hours prior to imaging. All images were acquired with identical microscope settings (60% brightness for FITC and 10% brightness for Hoechst). Fluorescence intensity of FITC in cells was quantified using ImageJ.

### Confocal Imaging of Cellular Uptake

Confocal microscopy uptake studies were conducted using Cy5-AS1411-conjugated nanoemulsions with FITC-labeled lipids. Images were acquired using a Nikon confocal microscope. Human triple negative breast cancer cells (MDA-MB-231) were plated for 48 hours at a density of 6,000 cells/dish in glass cell culture dishes (FluoroDish, World Precision Instruments, Sarasota, Fla. USA). The dishes were then treated with nanoemulsions (with or without AS1411) and incubated for 4 hr and 24 hr. Dishes were washed with HBSS, fixed with 3.5% paraformaldehyde for 15 minutes, stained with 0.05% Hoechst 33342 for 5 minutes at room temperature to detect nuclei, washed twice, and mounted (ClearMount, Invitrogen, Frederick, Md., USA) for at least 3 hours prior to imaging. All images were acquired with identical acquisition settings. The fluorescence intensity of FITC and Cy5 in cells was quantified using ImageJ.

### Flow Cytometry

Flow cytometry studies were performed using fluorescent nanoemulsions that were synthesized as described for confocal imaging. MDA-MB-231 and HCC1395 cells were plated in 12-well plates at a density of 16,000 cells/well for 24 hours. Cells were treated with fluorescent nanoemulsions (with or without AS1411) for 4 hr. Samples were then washed with PBS, trypsinized, washed by centrifugation, and analyzed with a flow cytometer (MACSQuant, Miltenyi Biotec). Data was analyzed using flow cytometry software (FlowJo).

### In Vitro Cytotoxicity Studies

Cytotoxicity of AS1411-conjugated drug-loaded nanoemulsions was tested in human lung cancer (A549) and breast cancer cells (MDA-MB-231 and HCC1395) using MTT assays. Control groups consisted of no treatment, untargeted drug-loaded nanoemulsions, or free drug. Nanoemulsions were added to cell cultures at various concentrations and incubated for 48 hr (breast cancer cells) or 72 hr (lung cancer cells). MTT results were normalized to the no treatment control samples.

### Results

### Characterization of Nanoemulsions

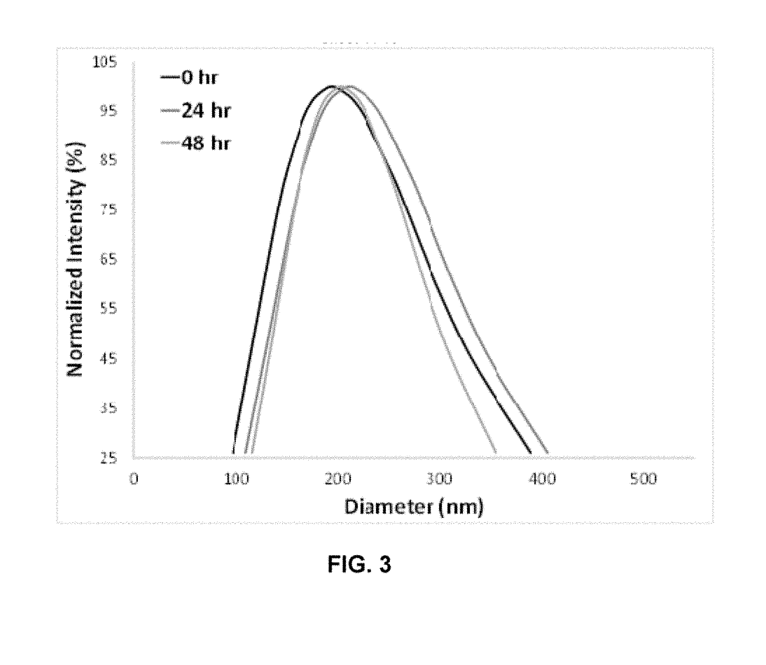

The size distribution of nanodroplet emulsions was determined using dynamic light scattering, indicating that the nanodroplets were stable for at least 48 hr when stored at 4° C. (FIG. 3). In addition, the loading efficiency of thymoquinone in nanodroplets was determined using absorbance spectrometry. The thymoquinone concentration in the nanodroplet emulsion after extrusion and 5-fold dilution in PBS was 1 mM, indicating a loading efficiency of 10%.

### Microscopy Imaging of Cellular Uptake

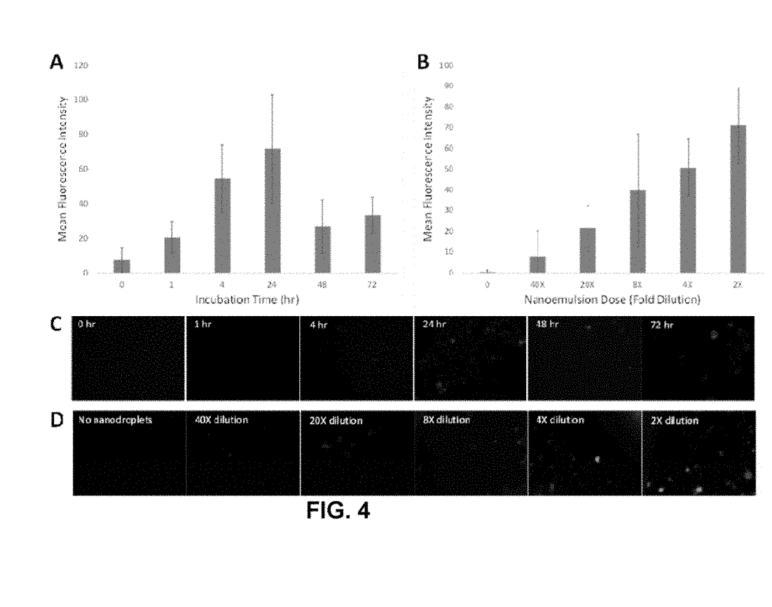

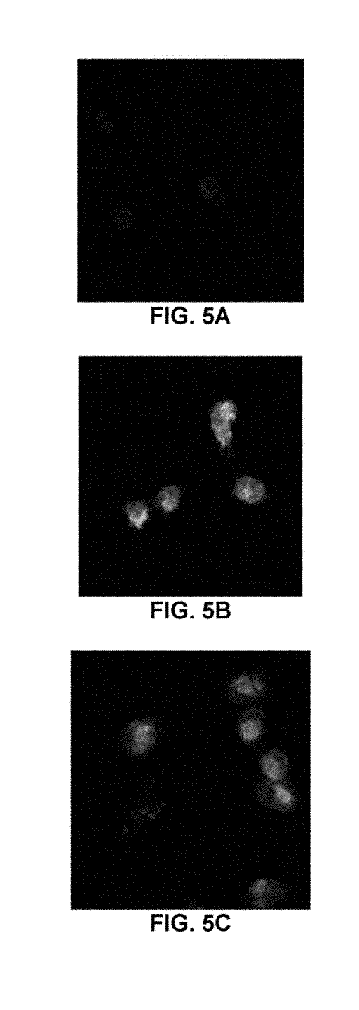

Fluorescence microscopy imaging was performed to assess uptake of fluorescent AS1411-conjugated nanodroplets by human lung cancer cells. At a 20× dilution, uptake was detected within 1 hr and persisted for at least 72 hr, with the peak fluorescence intensity detected at 24 hr (FIG. 4). Dose-dependent uptake was also observed at 72 hr (FIG. 4). Furthermore, confocal microscopy imaging indicated uptake and co-localization of fluorescent AS1411-conjugated nanoemulsions in human breast cancer cells (FIG. 5).

### Flow Cytometry Analysis

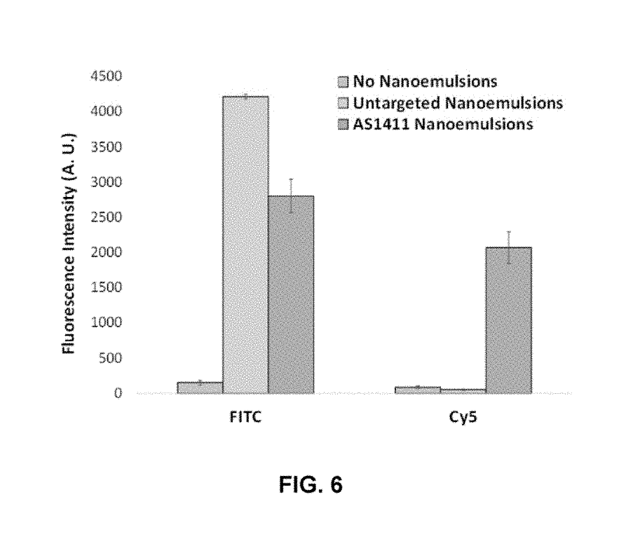

Flow cytometry was performed to assess uptake of fluorescent nanoemulsions by cancer cells. The fluorescence intensity of Cy5 (AS1411) and FITC (lipid) was significantly increased in cancer cells compared to negative control samples, indicating uptake of the nanoemulsions (FIG. 6).

### Nanoemulsion Cytotoxicity Studies

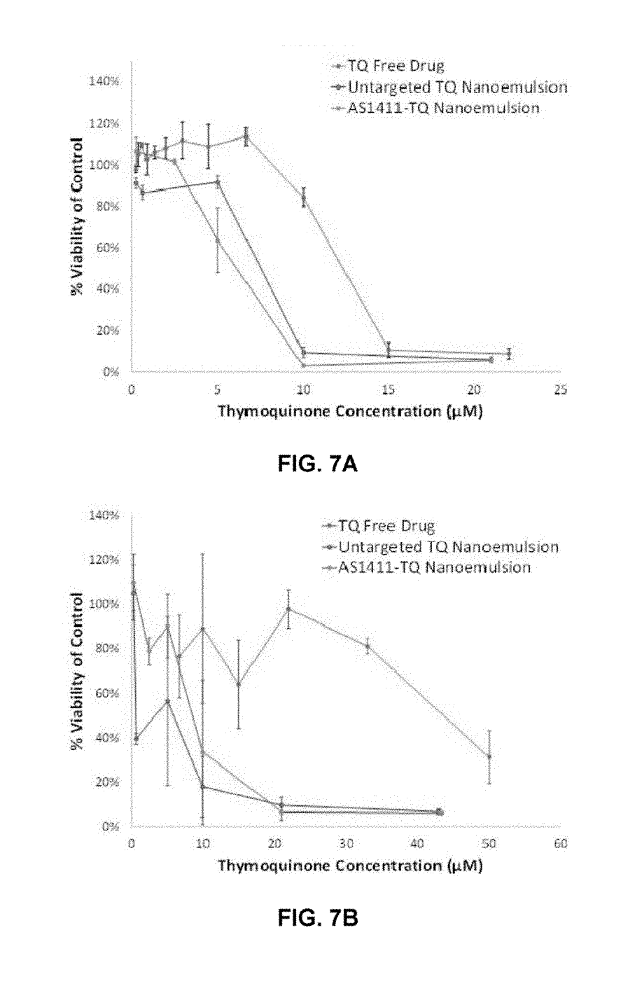

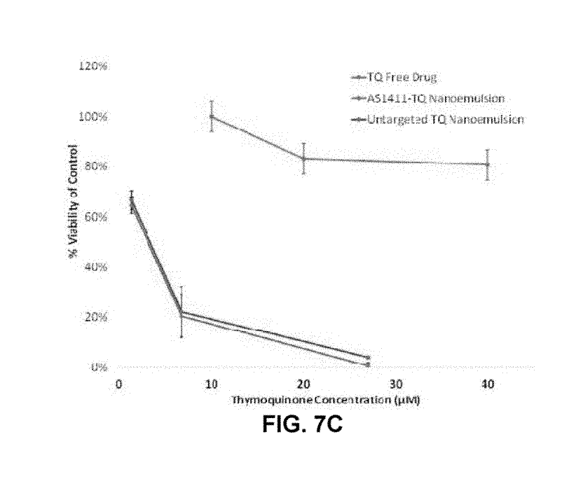

The effect of AS1411-conjugated nanodroplet emulsions on cytotoxicity at various concentrations was measured in three different cancer cell lines using MTT assays after 48 hr or 72 hr incubation (FIG. 7). There was a small increase in cytotoxicity with AS1411 targeting in two cell lines (MDA-MB-231 and A549) but not in one of the cell lines (HCC1395). However, in all three cell lines nanoemulsions significantly increased cytotoxicity of thymoquinone compared to free drug alone.

### Loading of Aptamers to Lipid Nanodroplets

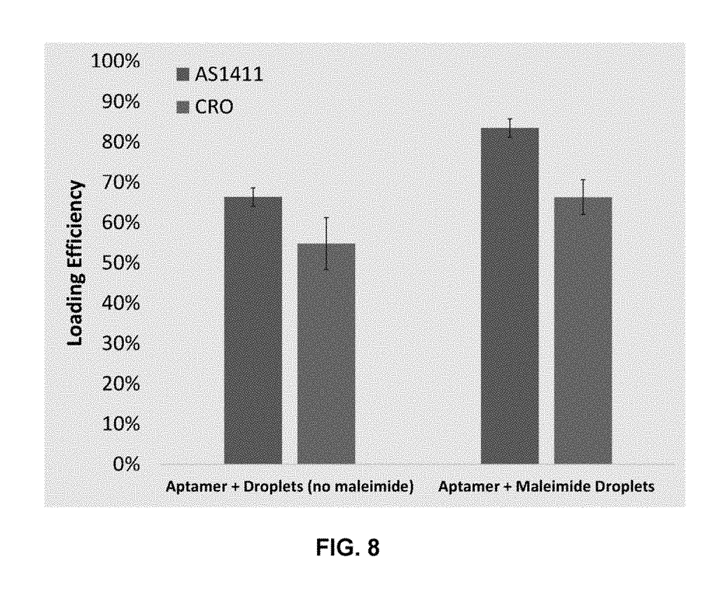

The aptamer loading efficiency of lipid nanodroplets with maleimide and lipid nanodroplets without maleimide was compared. AS1411 and CRO aptamers were attached to lipid nanodroplets. Aptamer loading efficiency was determined by measuring the absorbance at 260 nm of the supernatant after centrifugation of nanodroplets to detect unbound aptamer. Lipid nanodroplets without maleimide had an aptamer loading efficiency of 55-66%, whereas maleimide-lipid nanodroplets had an aptamer loading efficiency of 66-83%. These results are illustrated graphically in FIG. 8.

### Example 2

### (Prophetic)

Nanoemulsions, such as the nanoemolsion of Example 1, may be injected intravenously by bolus injection or infusion, into an animal model of cancer, or into a patient having cancer or a tumor. If ultrasound activation of nanoemulsions is desired, the subject will receive ultrasound treatment several hours later or the following day (4-24 hr after nanoemulsion infusion). This involves focusing the ultrasound beam on the tumor and applying short, high pressure (>1 MPa) bursts of ultrasound. The nanoemulsions will vaporize and release the drug at the site of the ultrasound focus (in the tumor). Alternatively, if a dye is present, then the perfluorocarbon in the nanoemulsions may be vaporized by application of light, such as infrared light, visible light, or UV light, to the tumor.

### REFERENCES

[1] Stedman, T. L. 2000. Stedman's medical dictionary. Lippincott Williams & Wilkins, Philadelphia. xxxvi, [127], 2098.

[2] Miller, D., P. Bates, and J. Trent. 2000. Antiproliferative activity of G-rich oligonucleotides and method of using same to bind to nucleolin, Int'l Pub. No. WO 00/61597.

[3] Bates P J, Miller D M, Trent J O, Xu X, “A New Method for the Diagnosis and Prognosis of Malignant Diseases” International Application, Int'l Pub. No. WO 03/086174 A2 (23 Oct. 2003).

[4] Bates P J, Miller D M, Trent J O, Xu X. “Method for the Diagnosis and Prognosis of Malignant Diseases” U.S. Patent App. Pub., Pub. No. US 2005/0053607 A1 (10 Mar. 2005).

[5] Derenzini M, Sirri V, Trere D, Ochs R L, “The Quantity of Nucleolar Proteins Nucleolin and Protein B23 is Related to Cell Doubling Time in Human Cancer Cells” Lab. Invest. 73:497-502 (1995).

[6] Bates P J, Kahlon J B, Thomas S D, Trent J O, Miller D M, “Antiproliferative Activity of G-rich Oligonucleotides Correlates with Protein Binding” J. Biol. Chem. 274:26369-77 (1999).

[7] Miller D M, Bates P J, Trent J O, Xu X, “Method for the Diagnosis and Prognosis of Malignant Diseases” U.S. Patent App. Pub., Pub. No. US 2003/0194754 A1 (16 Oct. 2003).

[8] Bandrnan O. Yue H. Corley N C, Shah P “Human Nucleolin-like Protein” U.S. Pat. No. 5,932,475 (3 Aug. 1999).

[9] Reyes-Reyes E M. Tang Y, Bates P J, “A New Paradigm for Aptamer Therapeutic AS1411 Action: Uptake by Macropinocytosis and Its Stimulation by a Nucleolin-Dependent Mechanism” Cancer Res 70(21): 8617-29 (2010).

[10] Perez-Herrero, A. Fernandez-Medarde, Advanced targeted therapies in cancer: Drug nanocarriers, the future of chemotherapy, Eur J Pharm Biopharm, 93 (2015) 52-79.

[11] C. Sawyers, Targeted cancer therapy, Nature, 432 (2004) 294-297.

[12] Tacar, P. Sriamornsak, C. R. Dass, Doxorubicin: an update on anticancer molecular action, toxicity and novel drug delivery systems, J Pharm Pharmacol, 65 (2013) 157-170.

[13] D. A. Fernandes, D. D. Fernandes, Y. Li, Y. Wang, Z. Zhang, D. Rousseau, C. C. Gradinaru, M. C. Kolios, Synthesis of Stable Multifunctional Perfluorocarbon Nanoemulsions for Cancer Therapy and Imaging, Langmuir, (2016).

[14] J. A. Kopechek, E. Park, C. S. Mei, N. J. McDannold, T. M. Porter,

Accumulation of phase-shift nanoemulsions to enhance MR-guided ultrasound-mediated tumor ablation in vivo, J Healthc Eng, 4 (2013) 109-126.

[15] N. Rapoport, Phase-shift, stimuli-responsive perfluorocarbon nanodroplets for drug delivery to cancer, Wiley Interdiscip Rev Nanomed Nanobiotechnol, 4 (2012) 492-510.

[16] R. Gupta, J. Shea, C. Scafe, A. Shurlygma, N. Rapoport, Polymeric micelles and nanoemulsions as drug carriers: Therapeutic efficacy, toxicity, and drug resistance, J Control Release. 212 (2015) 70-77.

[17] N. Rapoport, R. Gupta, Y. S. Kim, B. E. O'Neill, Polymeric micelles and nanoemulsions as tumor-targeted drug carriers: Insight through intravital imaging, J Control Release, 206 (2015) 153-160.

[18] J. P. Marshalek, P. S. Sheeran, P. Ingram, P. A. Dayton, R. S. Witte, T. O. Matsunaga, Intracellular delivery and ultrasonic activation of folate receptor-targeted phase-change contrast agents in breast cancer cells in vitro, J Control Release, 243 (2016) 69-77.

[19] C. H. Wang, S. T. Kang, Y. H. Lee, Y. L. Luo, Y. F. Huang, C. K. Yeh, Aptamer-conjugated and drug-loaded acoustic droplets for ultrasound theranosis, Biomaterials, 33 (2012) 1939-1947.

[20] J. A. Kopechek, E. J. Park, Y. Z. Zhang, Vykhodtseva, N. J. McDannold, T. M. Porter, Cavitation-enhanced MR-guided focused ultrasound ablation of rabbit tumors in viva using phase shift nanoemulsions, Phys Med Biol, 59 (2014) 3465-3481.

[21] O. D. Kripfgans, J. B. Fawkes, D. L. Miller, O. P. Eldevik, P. L. Carson, Acoustic droplet vaporization for therapeutic and diagnostic applications, Ultrasound Med Biol, 26 (2000) 1177-1189.

[22] E. Strohm, M. Rui, I. Gorelikov, N. Matsuura, M. Kolios, Vaporization of perfluorocarbon droplets using optical irradiation, Biomed Opt Express, 2 (2011) 1432-1442.

[23] K. Wilson, K. Homan, S. Emelianov, Biomedical photoacoustics beyond thermal expansion using triggered nanodroplet vaporization for contrast-enhanced imaging, Nat Commun, 3 (2012) 618.

[24] P. J. Bates, D. A. Laber, D. M, Miller, S. D. Thomas, J. O. Trent, Discovery and development of the G-rich oligonucleotide AS1411 as a novel treatment for cancer, Exp Mol Pathol, 86 (2009) 151-164.

[25] P. J. Bates, E. M. Reyes-Reyes, M. T. Malik, E. M. Murphy, M. G. O'Toole, J. O. Trent, G-quadruplex oliganucleotide AS1411 as a cancer-targeting agent: Uses and mechanisms, Biochim Biophys Acta, (2016).

[26] E. M. Reyes-Reyes, Y. Tang, P. J. Bates, A new paradigm for aptamer therapeutic AS1411 action: uptake by macropinocytosis and its stimulation by a nucleolin-dependent mechanism, Cancer Res, 70 (2010) 8617-8629.

[27] J. E. Rosenberg, R. M. Bambury, E. M. Van Allen, H. A. Drabkin, P. N. Lara, Jr., A. L. Harzstark, N. Wagle, R. A. Figlin, G. W. Smith, L. A. Garraway, T. Choueiri, F. Erlandsson, D. A. Later, A phase II trial of AS1411 (a novel nucleolin-targeted DNA aptamer) in metastatic renal cell carcinoma, Invest New Drugs, 32 (2014) 178-187.

[28] A. Latorre, C. Posch, Y Garcimartin, A. Celli, M. Sanlorenzo, I, Vujic, J. Ma, M. Zekhtser, K. Rappersberger, S. Ortiz-Urda, A. Somoza, DNA and aptamer stabilized gold nanoparticles for targeted delivery of anticancer therapeutics, Nanoscale, 6 (2014) 7436-7442.

[29] M. T. Malik, M. G. O'Toole, L. K. Casson, S. D. Thomas, G. T. Bardi, E. M. Reyes-Reyes, C. K. Ng, K. A. Kang, P. J. Bates, AS1411-conjugated gold nanospheres and their potential for breast cancer therapy, Oncotarget, 6 (2015)) 22270-22281.

[30] Y. S. Shiao, H. H. Chiu, P. H. Wu, Y. F. Huang, Aptamer-functionalized gold nanoparticles as photoresponsive nanoplatform for co-drug delivery, ACS Appl Mater Interfaces, 6 (2014) 21832-21841.

[31] M. Khader, P. M. Eckl, Thymoquinone: an emerging natural drug with a wide, range of medical applications. Iran J Basic Med Sci. 17 (2014) 960-957,

[32] C. C. Woo, A. P. Kumar, G. Sethi, K. H. Tan, Thymoquinorie: potential cure for inflammatory disorders and cancer, Biochem Pharmacol, 83 (2012) 443-451.

[33] L. Li, J. Hou, X. Liu, Y. Guo, Y. Wu, L. Zhang, Z. Yang. Nucleolin-targeting liposomes guided by aptamer AS1411 for the delivery of siRNA for the treatment of malignant melanomas, Biomaterials, 35 (2014) 3840-3850.

[34] Z. X. Liao, E. Y. Chuang, C. C. Lin, Y. C. Ho, K. J. Lin, P. Y. Cheng, K. J. Chen, H. J. Wei, H. W. Sung, An AS1411 aptamer-conjugated liposomal system containing a bubble-generating agent for tumor-specific chemotherapy that overcomes multidrug resistance, J Control Release, 208 (2015) 42-51.

[35] X. Li, Y. Yu, Q. Ji, L. Qiu, Targeted delivery of anticancer drugs by aptamer AS1411 mediated Pluronic F127/cyclodextrin-linked polymer composite micelles, Nanomedicine, 11 (2015) 176-184.

[36] J. Zhang, R. Chen, F. Chen, M. Chen, Y. Wang, Nucleolin targeting AS1411 aptamer modified pH-sensitive micelles: A dual-functional strategy for paclitaxel delivery, J Control Release, 213 (2015) e137-138.

[37] A. Aravind, P. Jeyamohan, R. Nair, S. Veeranarayanan, Y. Nagaoka, Y. Yoshida, T. Maekawa, D. S. Kumar, AS1411 aptamer tagged PLGA-lecithin-PEG nanoparticles for tumor cell targeting and drug delivery, Biotechnol Bioeng, 109 (2012) 2920-2931.

[38] J Wu, C. Song, C Jiang, X. Shen, Q. Qiao, Y. Hu, Nucleolin targeting AS1411 modified protein nanoparticle for antitumor drugs delivery, Mol Pharm, 10 (2013) 3555-3563.

[39] W. J. Duncanson, L. R. Arriaga, W. L. Ung, J. A. Kopechek, T. M. Porter, D. A. Weitz, Microfluidic fabrication of perfluorohexane-shelled double emulsions for controlled loading and acoustic-triggered release of hydrophilic agents, Langmuir. 30 (2014) 13765-13770.

[40] M. L. Fabiilli, K. J. Haworth, I. E. Sebastian, O. D. Kripfgans, P. L. Carson, J. B. Fowlkes, Delivery of chlorambucil using an acoustically-triggered perfluoropentane emulsion, Ultrasound Med Biol, 36 (2010) 1364-1375.

[41] Kopechek J A, Park E, Mei C S, McDannold N J and Porter T M 2013 Accumulation of phase-shift nanoemulsions to enhance MR-guided ultrasound-mediated tumor ablation in vivo J. Healthc, Eng. 4 109-26.

[42] Kopechek J A, Park E, Zhang Y, Vykhodtseva N I, McDannold N J and Porter T M, 2014 Cavitation-enhanced MR-guided focused ultrasound ablation of rabbit tumors in vivo using phase shift nanoemulsions Phys. Med. Biol. 59 (2014) 3465-3481.

[43] Kopechek, J. A., Zhang, P., Burgess, M. T., Porter, T. M. Synthesis of Phase-shift Nanoemulsions with Narrow Size Distributions for Acoustic Droplet Vaporization and Bubble-enhanced Ultrasound-mediated Ablation. J. Vis. Exp. (67), e4308, DOI: 10.3791/4308 (2012).

[44] U.S. Pat. No. 9,260,517.

[45] Bates, et al., “Discovery and Development of the G-rich Oligonucleotide AS1411 as a Novel Treatment for Cancer”, Experimental and Molecular Pathology, Vol. 86, pp. 151-164 (2009).

[46] Malik, et al., “AS1411-conjugated gold nanospheres and their potential for breast cancer therapy”, Oncotarget, Vol. 6, pp. 22270-22281 (2015).

[47] Wang, et al., “Aptamer-conjugated and drug-loaded acoustic droplets for ultrasound theranosis”, Biomaterials, Vol. 33, pp. 1939-1947 (2012).

1. A micelle, comprising:
a first phospholipid,
a second phospholipid,
a targeting agent, conjugated to the first phospholipid,
a perfluorocarbon, and
a therapeutically active compound,
wherein the first phospholipid and the second phospholipid form a shell enclosing the perfluorocarbon and the therapeutically active compound,
the targeting agent comprises an anti-nucleolin agent, and
the therapeutically active compound comprises a chemotherapeutic agent.

2. An emulsion, comprising:
a plurality of the micelles of claim 1, and
a continuous aqueous phase.

3. The emulsion of claim 2, wherein the micelles have an average diameter of 50-500 nm.

4. The emulsion of claim 3, wherein the micelles have an average diameter of 150-250 nm.

5. The micelle of claim 1, wherein the perfluorocarbon is perfluoropentane.

6. The micelle of claim 1, further comprising a dye.

7. The micelle of claim 1, wherein the anti-nucleolin agent comprises an anti-nucleolin oligonucleotide.

8. The micelle of claim 1, wherein the anti-nucleolin agent comprises AS1411.

9. The micelle of claim 1, wherein the anti-nucleolin agent comprises an antibody.

10. The micelle of claim 1, wherein the anti-nucleolin agent comprises a nucleolin targeting protein.

11. The micelle of claim 1, wherein the therapeutically active compound comprises the chemotherapeutic agent.

12. The micelle of claim 1, wherein the therapeutically active compound comprises the chemotherapeutic agent, and
the micelle comprises a second chemotherapeutic agent.

13. The micelle of claim 1, wherein the therapeutically active compound is hydrophobic.

14. The micelle of claim 1, wherein the therapeutically active compound is thymoquinone or paclitaxel.

15. The micelle of claim 1, wherein the targeting agent is conjugated to the first phospholipid via a thiol-maleimide linkage.

16. A pharmaceutical composition for treating cancer, comprising the emulsion of claim 2, and a pharmaceutically acceptable carrier.

17. A method, of treating cancer, comprising administering an effective amount of the pharmaceutical composition of claim 16, to a patient in need thereof.

18. A method of treating cancer, comprising:
administering an effective amount of the pharmaceutical composition of claim 16, to a patient in need thereof, and
administering high-intensity focused ultrasound to the patient, to the cancer or to a tumor.

19. (canceled)

20. (canceled)

21. A method of imaging cancer or a tumor, comprising:
(1) administering the emulsion of claim 2 to a patient, and
(2) imaging the cancer or tumor, with ultrasound.

22. A micelle, comprising:
a first phospholipid,
a second phospholipid,
a targeting agent, conjugated to the first phospholipid,
a perfluorocarbon, and
a therapeutically active compound,
wherein the first phospholipid and the second phospholipid form a shell enclosing the perfluorocarbon and the therapeutically active compound,
the targeting agent comprises an anti-nucleolin agent, and
the therapeutically active compound comprises a cytotoxic agent.


SEQUENCE LISTING (38 sequences)

SEQ ID NO: 1 - DNA, 29 bp, Artificial Sequence
  Description of Artificial Sequence Synthetic polynucleotide sequence
tttggtggtggtggttgtggtggtggtgg

SEQ ID NO: 2 - DNA, 29 bp, Artificial Sequence
  Description of Artificial Sequence Synthetic polynucleotide sequence
tttggtggtggtggttttggtggtggtgg

SEQ ID NO: 3 - DNA, 29 bp, Artificial Sequence
  Description of Artificial Sequence Synthetic polynucleotide sequence
tttggtggtggtggtggtggtggtggtgg

SEQ ID NO: 4 - DNA, 29 bp, Artificial Sequence
  Description of Artificial Sequence Synthetic polynucleotide sequence
tttggtggtggtggtttgggtggtggtgg

SEQ ID NO: 5 - DNA, 13 bp, Artificial Sequence
  Description of Artificial Sequence Synthetic polynucleotide sequence
tggtggtggtggt

SEQ ID NO: 6 - DNA, 14 bp, Artificial Sequence
  Description of Artificial Sequence Synthetic polynucleotide sequence
ggtggttgtggtgg

SEQ ID NO: 7 - DNA, 15 bp, Artificial Sequence
  Description of Artificial Sequence Synthetic polynucleotide sequence
gttgtttggggtggt

SEQ ID NO: 8 - DNA, 15 bp, Artificial Sequence
  Description of Artificial Sequence Synthetic polynucleotide sequence
ttggggggggtgggt

SEQ ID NO: 9 - DNA, 25 bp, Artificial Sequence
  Description of Artificial Sequence Synthetic polynucleotide sequence
ggttggggtgggtggggtgggtggg

SEQ ID NO: 10 - DNA, 26 bp, Artificial Sequence
  Description of Artificial Sequence Synthetic polynucleotide sequence
ggtggtggtggttgtggtggtggtgg

SEQ ID NO: 11 - DNA, 28 bp, Artificial Sequence
  Description of Artificial Sequence Synthetic polynucleotide sequence
tttggtggtggtggttgtggtggtggtg

SEQ ID NO: 12 - DNA, 28 bp, Artificial Sequence
  Description of Artificial Sequence Synthetic polynucleotide sequence
tttggtggtggtggtgtggtggtggtgg

SEQ ID NO: 13 - DNA, 29 bp, Artificial Sequence
  Description of Artificial Sequence Synthetic polynucleotide sequence
ggtggtggtggttgtggtggtggtggttt

SEQ ID NO: 14 - DNA, 32 bp, Artificial Sequence
  Description of Artificial Sequence Synthetic polynucleotide sequence
ggtggttgtggtggttgtggtggttgtggtgg

SEQ ID NO: 15 - DNA, 32 bp, Artificial Sequence
  Description of Artificial Sequence Synthetic polynucleotide sequence
tttggtggtggtggttgtggtggtggtggttt

SEQ ID NO: 16 - DNA, 56 bp, Artificial Sequence
  Description of Artificial Sequence Synthetic polynucleotide sequence
ggtggtggtggttgtggtggtggtggttgtggtggtggtggttgtggtggtggtgg

SEQ ID NO: 17 - DNA, 35 bp, Artificial Sequence
  Description of Artificial Sequence Synthetic polynucleotide sequence
tcgagaaaaactctcctctccttccttcctctcca

SEQ ID NO: 18 - DNA, 29 bp, Artificial Sequence
  Description of Artificial Sequence Synthetic polynucleotide sequence
tttcctcctcctccttctcctcctcctcc

SEQ ID NO: 19 - DNA, 24 bp, Artificial Sequence
  Description of Artificial Sequence Synthetic polynucleotide sequence
ttagggttagggttagggttaggg

SEQ ID NO: 20 - DNA, 11 bp, Artificial Sequence
  Description of Artificial Sequence Synthetic polynucleotide sequence
ggtggtggtgg

SEQ ID NO: 21 - DNA, 14 bp, Artificial Sequence
  Description of Artificial Sequence Synthetic polynucleotide sequence
ggtggttgtggtgg

SEQ ID NO: 22 - DNA, 15 bp, Artificial Sequence
  Description of Artificial Sequence Synthetic polynucleotide sequence
ggttggtgtggttgg

SEQ ID NO: 23 - DNA, 10 bp, Artificial Sequence
  Description of Artificial Sequence Synthetic polynucleotide sequence
gggttttggg

SEQ ID NO: 24 - DNA, 20 bp, Artificial Sequence
  Description of Artificial Sequence Synthetic polynucleotide sequence
ggttttggttttggttttgg

SEQ ID NO: 25 - DNA, 15 bp, Artificial Sequence
  Description of Artificial Sequence Synthetic polynucleotide sequence
ggttggtgtggttgg

SEQ ID NO: 26 - DNA, 12 bp, Artificial Sequence
  Description of Artificial Sequence Synthetic polynucleotide sequence
ggggttttgggg

SEQ ID NO: 27 - DNA, 10 bp, Artificial Sequence
  Description of Artificial Sequence Synthetic polynucleotide sequence
gggttttggg

SEQ ID NO: 28 - DNA, 28 bp, Artificial Sequence
  Description of Artificial Sequence Synthetic polynucleotide sequence
ggggttttggggttttggggttttgggg

SEQ ID NO: 29 - DNA, 24 bp, Artificial Sequence
  Description of Artificial Sequence Synthetic polynucleotide sequence
ttggggttggggttggggttgggg

SEQ ID NO: 30 - DNA, 16 bp, Artificial Sequence
  Description of Artificial Sequence Synthetic polynucleotide sequence
gggtgggtgggtgggt

SEQ ID NO: 31 - DNA, 26 bp, Artificial Sequence
  Description of Artificial Sequence Synthetic polynucleotide sequence
ggttttggttttggttttggttttgg

SEQ ID NO: 32 - DNA, 29 bp, Artificial Sequence
  Description of Artificial Sequence Synthetic polynucleotide sequence
tttcctcctcctccttctcctcctcctcc

SEQ ID NO: 33 - DNA, 26 bp, Artificial Sequence
  Description of Artificial Sequence Synthetic polynucleotide sequence
cctcctcctccttctcctcctcctcc

SEQ ID NO: 34 - DNA, 6 bp, Artificial Sequence
  Description of Artificial Sequence Synthetic polynucleotide sequence
tggggt

SEQ ID NO: 35 - DNA, 7 bp, Artificial Sequence
  Description of Artificial Sequence Synthetic polynucleotide sequence
gcatgct

SEQ ID NO: 36 - DNA, 11 bp, Artificial Sequence
  Description of Artificial Sequence Synthetic polynucleotide sequence
gcggtttgcgg

SEQ ID NO: 37 - DNA, 4 bp, Artificial Sequence
  Description of Artificial Sequence Synthetic polynucleotide sequence
tagg

SEQ ID NO: 38 - DNA, 23 bp, Artificial Sequence
  Description of Artificial Sequence Synthetic polynucleotide sequence
ggggttggggtgtggggttgggg